(19) United States
(12) Patent Application Publication
(10) Pub. No.: US 2009/0299250 A1
(43) Pub. Date: Dec. 3, 2009

(54) ANTIVIRAL FILTER AND ITS USE IN AN AIR PURIFIER, AIR CONDITIONER OR AIR HUMIDIFIER

(75) Inventors: Quang Trong Nguyen, Le Havre (FR); Laurent Lebrun, Darnetal (FR)

Correspondence Address: YOUNG & THOMPSON, 209 Madison Street, Suite 500, ALEXANDRIA, VA, 22314, US

(73) Assignee: Universite De Rouen, Mont-Saint-Aignan Cedex (FR)

(21) Appl. No.: 12/374,145

(22) PCT Filed: Jul. 19, 2007
(86) PCT No.: PCT/FR2007/051691

§ 371 Date: Apr. 15, 2009

(30) Foreign Application Priority Data
Jul. 19, 2006 (FR) 0606591

(51) Int. Cl. A61L 15/14; B32B 5/02; B05D 3/00; B01D 39/14; A62B 7/10

(52) U.S. Cl. 602/43; 442/59; 427/385.5; 55/524; 128/206.12

(57) ABSTRACT

Fiber bed, preferably negatively charged, covered with a cationic polymer having a positive net charge; antiviral filter comprising said fiber bed; and method for the fabrication of said fiber bed having antiviral characteristic.

The present invention relates to the field of health risk and public health, in particular linked to the presence of airborne viruses. The invention relates more particularly to an antiviral and optionally antibacterial filter which is capable of sequestering the viruses and optionally the bacteria present in the ambient air. The invention also relates to all the products comprising said filter such as for example air conditioners, air purifiers, air humidifiers, or medical devices such as for example surgical masks.

Eliminating airborne viruses is nowadays an important public health issue. For example, the recent viral epidemics in Asia (avian influenza, SARS) have shown that there is a real need for protection when viral attacks occur. Immunization is a relatively inefficient solution, due to the rapid mutations of the viruses, their sudden multiplication and propagation. Existing bacterial filters, such as the HEPA filters present for example in conventional surgical masks, are not an efficient protective barrier against viruses. These filters are actually made up of layers of non-woven polypropylene, the pores of which having a size of 0.2 μm. This pore size is not very efficient against viruses since the latter are smaller in size (from 80 to 110 nm). Manufacturing antiviral filters may thus represent an interesting solution in response to this need for protection.

Several approaches to the production of antiviral filters have been described.

For example, Japanese Patent Application JP2004432430 describes a filter containing a compound extracted from Sasa veitchii grass, which may have antibacterial and antiviral properties.

U.S. Pat. No. 5,888,527 discloses an antiviral mask impregnated with tea polyphenol containing catechins and theaflavins, which may make it possible to inactivate viruses by inhibiting viral replication and by altering the physical properties of the viral membranes.

The international patent application WO03/051460 relates to a mask comprising a passive filter and a disinfectant active filter. The passive filter may allow retention of dust particles, bacteria and spores, whilst the active filter may kill bacteria, spores and viruses, the size of which being too small to be blocked by the passive filter. The active filter comprises antibacterial, antibiotic or antiviral agents such as chlorohexidine or other antiseptic compounds containing chlorine or an antiseptic halogen.

Finally, Kawabata et al. particularly addressed 4-vinylpyridine polymers, and demonstrated that these polymers had an antibacterial activity (Kawabata et al., Antibacterial activity of soluble pyridinium-type polymer, Applied and Environmental Microbiology, 1988, pp. 2532-2535), and also an antiviral activity when they are polymerized in the form of beads having a diameter of 1.7 microns in a nonwoven fabric (N. Kawabata et al. 1998, Reactive & functional polymers, No. 37 p 213-218). In this publication, the 4-vinylpyridine polymer is polymerized on a nonwoven membrane having a pore size of 14 μm, in presence of divinylbenzene, in order to obtain beads having a diameter of 1.7 microns. According to the authors, the presence of divinylbenzene is essential, as in its absence, polymerization leads to a film of 4-vinylpyridine homopolymers which makes it difficult to obtain a microporous membrane. It is thus essential for the 4-vinylpyridine to be polymerized in the form of beads in order to obtain a microporous membrane.

According to Kawabata et al., the effectiveness of the viral retention may result from the specific affinity of this particular pyridine type polymer (N-benzyl-4-vinylpyridinium polychloride) for the viruses. Without being able to explain the causes of this affinity, Kawabata puts forward the hypothesis that (1) the electrostatic interactions between the positive charges of the polymer and the negative charges of the viruses and (2) the hydrophobic interactions between this polymer and the viruses, could play significant roles in this specific affinity. It is to be emphasized that, as there is no control test, this publication does not make it possible to identify whether or not the observed viral retention is actually due to the 4-vinylpyridine polymer or to the viral loss due to the experimental conditions.

Recently, Patent Application WO2006/071191 described an antimicrobial and/or antiviral product, coated with a polymer modified from a precursor polymer, said precursor polymer being selected from the group of polymers having the general formula I, II or III or copolymers of the latter:

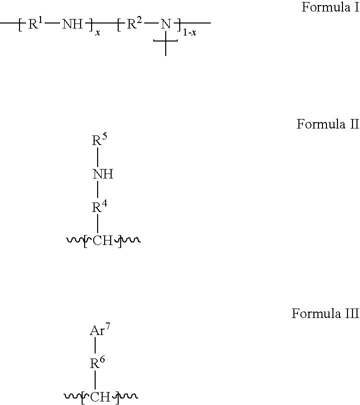

wherein:

R¹ and R² independently are selected from linear or branched (C₁-C₆) hydrocarbon chains,

X is within the range from 0 to 1,

R⁴ is selected from a direct bond and a linear or branched (C₁-C₆) hydrocarbon chain,

R⁵ is selected from hydrogen or linear or branched (C₁-C₆) hydrocarbon chains,

R⁶ is selected from a direct bond or linear or branched (C₁-C₆) hydrocarbon chains,

Ar⁷ is a hetero-aromatic group including a nitrogen atom,

and wherein said precursor polymer is modified in that:
at least some of said nitrogen atoms are substituted with a substituent selected from the group consisting of linear or branched C₁-C₂₀ alkyl groups, and
at least some of the nitrogen atoms in said precursor polymer are quaternized.

Within the meaning of the invention described in WO2006/071191, the expression “polymer with quaternized nitrogen atoms” refers to compounds of the following general formula:

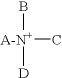

wherein at least one of the residues “A”, “B”, “C” and “D” forms part of the polymer repeat unit, and wherein the residue or residues “A”-“D” not included in the polymer repeat unit are any residues forming with the nitrogen a stable cationic quaternary compound formed in a covalent fashion. The term non-covalent within the meaning of WO2006/071191 refers to a non-covalent bond such as a hydrogen bond.

The Applicant has sought to develop a filter having an antiviral activity, said filter having a small pore size, preferably less than 5 μm, very preferably less than 1 μm and still more preferably less than or equal to 0.2 μm. The filter according to the invention may be prepared according to a simple method which requires no polymerization in situ, said filter having a non-occlusive surface, which means that the fibre membrane or bed remains microporous.

The Applicant thus established that the affinity between a polymer and a virus is due to the density of the positive charges distributed through the polymer network situated over the whole surface of the fibre bed, said charges preferably being borne by amine functions situated in the main chain and/or in the side chains of the polymer.

Thus, one goal of the present invention is to propose an alternative solution to existing antiviral filters in order to meet the need for protection against airborne viruses, and the invention relates to the use of a cationic polymer preferably having more than one amine function per unit, capable of being deposited on a fibre bed, preferably non-woven, as a virus trap.

The present invention also relates to a fibre bed, preferably negatively charged, coated with at least one cationic polymer preferably having more than one amine function per unit, the nitrogen atoms of which being not substituted by alkyl groups and the nitrogen atoms of which being not quaternized, and having a protonation level of at least 20%.

By fibre bed is meant any woven or nonwoven layer composed of fibres made of natural, artificial or synthetic polymers, for example cellulose or non-cellulose fibres such as polyester, polyethylene, polypropylene or polyamide fibres. According to a preferred embodiment of the invention, the fibre bed is microporous, i.e. it has a small pore size, preferably less than 5 μm, very preferably less than 1 μm and still more preferably less than or equal to 0.2 μm.

By cationic polymer is meant any protonable polymer, preferably of the type comprising amine functions on its main chain and/or on its side chain or chains, and having a protonation level such that it is capable of binding to a fibre bed on the one hand and of binding viruses on the other hand. Preferably, the protonation level of a cationic polymer is at least 20%.

The cationic polymer of the present invention is therefore different from that described in WO2006/071191 by the fact that it comprises no nitrogen atoms substituted by alkyl groups nor quaternized nitrogen atoms within the meaning of WO2006/071191. The cationic polymer of the present invention comprises amine functions on its main chain and/or on its side chain or chains, and has a protonation level of at least 20%.

Within the meaning of the present invention, the term “protonated amine” or “protonated nitrogen” refers to compounds of the following general formula:

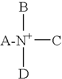

wherein at least one of the residues “A”, “B”, “C” and “D” forms part of the polymer repeat unit, and wherein the residue or residues “A”-“D” not included in the polymer repeat unit are hydrogen atoms forming with the nitrogen a stable cationic quaternary compound formed in a non-covalent fashion.

By “protonation level” is meant the percentage of nitrogen atoms which are protonated.

In an embodiment of the invention, said fibre bed thus coated has a charge density of 1×10⁻⁶ to 1×10⁻⁸ mole of charge per cm² of fibre bed or 1×10⁻⁵ to 1×10⁻³ meq per cm² of fibre bed, preferably said fibre bed has a charge density of 1×10⁻⁷ mol of charge per cm² of bed or 1×10⁻⁴ meq per cm² of fibre bed.

The assay of the charge density per cm² of fibre bed is carried out by the methods known to a person skilled in the art such as those making it possible to determine the exchange capacity of a membrane or of an ion exchange resin described by F. Helfferich, Ion Exchange. McGraw-Hill Book Company Inc. Ed. (1962), Chapter 4; pages 72-94.

According to an embodiment, the polymer comprises cationic groups on its main chain, and/or on its side chain or chains.

In a first embodiment, said cationic polymer comprises a polymer bearing amine groups (primary, secondary, tertiary amine groups) on its main chain, at least part of the amine groups, advantageously at least 20%, preferably at least 30%, very preferably at least 50%, of the amine groups being protonated. Preferably, these cationic polymers are polydimethylamine-co-epichlorhydrin or polydimethylamine-co-epichlorhydrin-co-ethylenediamine. The assay of the percentage of protonation is carried out by techniques well known to a person skilled in the art such as that described by Eyler et al. (Eyler R W, Krug T S, Siephius S, Analytical Chemistry 1947; 19(1) 24-7). Other techniques making it possible to assay the percentage of protonation are described by Muller et al. (G Muller, C Ripoll and E Selegny, European Polymer Journal, volume 7 (10), 1971, pages 1373-1392).

In a second embodiment, the cationic polymer comprises a polymer bearing amine groups (primary, secondary, tertiary) on its side chain or chains, at least part of the amine groups, advantageously at least 20%, preferably at least 30%, very preferably at least 50%, of the amine groups being protonated. Preferably, these cationic polymers are polyvinylamine, modified polyacrylamide, polyvinylimidazole, diethylaminoethyl polysaccharides, chitosan, polymethacrylate, preferably poly 2-dimethylaminoethyl-methacrylate, and do not include 4-vinylpyridine.

In a very preferred third embodiment, the cationic polymer comprises a polymer bearing (primary, secondary, tertiary) amine groups on its main chain and on its side chain or chains, at least part of the amine groups, and advantageously at least 20%, preferably at least 30%, very preferably at least 50%, of the amine groups being protonated. Preferably, these cationic polymers are polyethylene-imine.

According to a preferred embodiment of the invention, said polymer is polyethlene-imine.

The present invention also relates to an antiviral and optionally antibacterial filter comprising at least one fibre bed coated with at least one cationic polymer as described previously.

In a preferred embodiment, the filter of the invention is an antiviral and antibacterial filter, and comprises at least one fibre bed coated with a cationic polymer as described previously, said fibre bed having a pore size equal to or less than 0.2 μm. The pore size equal to or less than 0.2 μm confers an antibacterial property on the fibre bed; the surface of this fibre bed is then coated by at least one cationic polymer, the fibre bed thus acquires an antiviral property in addition to the antibacterial property.

By antiviral properties of the fibre bed is meant the ability of said fibre bed to sequester viruses.

By airborne virus is meant in particular the influenza virus (Influenza virus A, B and C), or the coronavirus (for example SARS-CoV). Other viruses also concerned by the invention are in particular the variola virus, the bacteriophages (E. Coli), the hepatitis virus, the poliovirus, rotavirus, the tobacco mosaic virus, the ebola virus and other infectious viruses.

In another preferred embodiment, the filter of the invention is an antiviral and antibacterial filter, and comprises at least one fibre bed coated with a cationic polymer as described previously, and at least one fibre bed having a pore size equal to or less than 0.2 μm.

Advantageously, the fibre bed according to the invention is effective for a period of at least 5 hours, preferably at least 12 hours and very preferably at least 18 hours.

A subject of the present invention is also an air purifier, an air-conditioner or an air humidifier comprising at least one antiviral and optionally antibacterial filter as described previously.

A subject of the present invention is also a medical device comprising at least one antiviral and optionally antibacterial filter as described previously, in particular a surgical mask, dressings, protective garments such as overalls etc.

The present invention also relates to a method for the manufacturing of a fibre bed with antiviral properties, comprising contacting said fibre bed with a solution of cationic polymer as described previously, then a drying stage.

During the contact of the cationic polymer with the fibre bed, the cationic functions, in particular quaternary amine, may interact with the fibre bed, which leads to the binding of the polymer onto the fibre bed.

According to an embodiment, the cationic polymer is solubilized in a solvent or a solvent mixture. In a preferred embodiment, the solvent is a polar solvent, preferably water.

Very preferably, the cationic polymer is solubilized in an ionizable salt, in particular NaCl.

According to an embodiment, the concentration of the polymer in said cationic polymer solution is from 0.1 to 200 g.l⁻¹, preferably from 1 to 100 g.l⁻¹, more preferably from 20 to 80 g.l⁻¹ and very preferably from 40 to 50 g.l⁻¹.

According to an embodiment, the pH of the polymer solution is preferably acid, preferably from 3 to 6.5, and very preferably the pH of the polymer solution is 6.

According to an embodiment, the fibre bed is soaked in the polymer solution at ambient temperature or the polymer solution is sprayed onto the fibre bed at ambient temperature.

According to an embodiment, the fibre bed is soaked in the polymer solution for 1 minute to 3 hours, preferably for 5 minutes to 1 hour, very preferably for 10 to 30 minutes.

According to an embodiment, the fibre bed is washed with water before drying in order to eliminate the excess of polymer solution.

According to an embodiment, the fibre bed is left to dry at ambient temperature by evaporation or ventilated with air. The drying stage can be accelerated by passage through a ventilated or vacuum oven.

According to an embodiment, the thickness of the polymer film obtained on the surface of the fibre bed is from 0.5 10⁻³ μm to 5 μm, preferably from 0.001 to 1 μm, very preferably from 0.001 to 0.01 μm.

The present invention comprises a method for the manufacturing of a fibre bed with antiviral properties as described previously further comprising a stage of cross-linking the polymer after drying. This optional operation sometimes makes it possible to optimize the binding of the polymer to the fibres.

The present invention also comprises a method for the manufacturing of a fibre bed with antiviral and antibacterial properties wherein the method for the manufacturing of a fibre bed with antiviral properties as described previously is applied to a fibre bed comprising a pore size equal to or less than 0.2 μm.

The present invention will be better understood with the help of the following additional description, which refers to examples for manufacturing the fibre bed with antiviral properties of the invention.

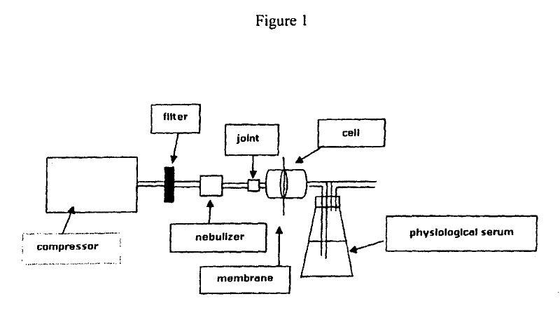

In the following examples, given by way of illustration, reference is made to the attached FIG. 1, which illustrates the assembly allowing the filtration experiments.

1—Materials and Methods

Reagents and Supports

A branched polyethylene-imine (PEI) with a high molecular weight obtained from Aldrich Chemicals (reference 40, 872-7, batch 05906DU-202) was chosen for these experiments for obtaining a fibre bed with antiviral properties.

The fibre bed used in these experiments originates from the third layer of surgeons' FFP1 rectangular masks. Infra-Red analysis in ATR mode has revealed that the first two layers of a mask (towards the outside) are constituted by nonwoven polypropylene and serve as bacterial filters, whereas the third layer, also nonwoven (towards the face) is constituted by a mixture of polyester-cellulose or esterified cellulose. These layers are negatively charged.

Assay of the Protonated Amine Groups

This measurement is carried out by pH-metric assay.

25 ml of a 0.44% PEI solution (4.4 g.l⁻¹ or 0.102 mol.l⁻¹) in 0.2 mol.l⁻¹ NaCl are assayed with a 0.1 mol l⁻¹ HCl solution,

The pH of the initial solution (non-protonated PEI) is 11.

14 ml of 0.1 mol.l⁻¹ HCl were added for a total volume of 18 ml in order to reach a pH=6.

At pH=6, the protonation level of the PEI is 75%±5% (14 ml/18 ml).

Assay of the Charge Density per cm² of Fibre Bed.

A precise surface area of the fibre bed (232 cm²) is soaked for 12 hours in 50 ml of a 4.4% PEI solution in 0.2 mol.l⁻¹ NaCl. The fibre bed is then placed in a 0.1 mol.l⁻¹ HCl solution in order to protonate all binded PEI.

The fibre bed is then washed with water until a constant pH is reached, in order to draw off the excess HCl.

The fibre bed is finally placed in 8 ml of water. The quantity of positive charge present on the fibre bed is assayed with 0.01 mol.l⁻¹ soda.

The initial pH is 8.56. An equivalent volume of 2.5 cm³ of soda 0.01 mol.l⁻¹ is found.

This corresponds to a concentration of 3.125×10⁻³ mol/l of acid, or 2.5×10⁻⁵ mole of acid in the 8 ml of water.

Considering that one mole of acid had reacted with one mole of PEI repeat unit (M_{PEI}=43 g.mol⁻¹), 1.075×10⁻³ g of PEI are therefore binded to the 232 cm² of fibre bed, which is equivalent to 4.63×10⁻⁶ g of PEI per cm² of fibre bed.

The charge density value of the fibre bed is therefore 1.1×10⁻⁷ mole of charge per cm² of fibre bed or, better, 1.1×10⁻⁴ meq per cm² of fibre bed.

Modification of Layer 3

The functionalization relates to layer 3 of the mask, hereafter called the fibre bed.

The nonwoven layer is soaked for one day in a PEI solution, 4.4% by mass, in 0.2 M NaCl at pH=8 or pH=6.

The salt makes it possible to increase the quantity of binded polymer. The pH value of 8 corresponds to a previously determined optimum pH for binding. The pH value of 6 makes it possible to increase the protonation, preferably the quaternization of the PEI (7<pKa<9) and therefore to improve its binding. The layer is then drained, washed with water under slow stirring for 10 hours, then air-dried. Finally, the layer is sterilized under UV before being mounted in the filtration cell.

Virus and Bacterial Strain

The bacteriophage T4 which is a parasite of the strain E. coli was chosen as test virus. The bacteriophage T4 and the E. coli strain are both non-pathogenic for humans and for the environment.

Culture Media

Medium 1 serves for infecting E. coli with the phage and for carrying out the filtration experiment. Its composition comprises one litre of deionized water (Milliro®)): peptone and meat extract: 13 g, yeast extract: 5 g; NaCl: 5 g; KH₂PO₄: 8 g; NaOH in order to obtain a pH of 7.6.

Medium 2, richer than the first, serves for assaying the phage. Its composition comprises one litre of deionized water (Milliro®): peptone and meat extract: 10 g; glucose: 10 g; NaCl: 3 g, KH₂PO₄: 0.044 g; CaCl₂: 0.011 g; MgCl₂: 0.203 g; NaOH in order to obtain a pH of 7.6.

The peptone, meat and yeast extracts are substrates rich in proteins, glucides and water-soluble vitamins which are used for bacterial growth. The glucose is a glucide which has the same role. NaCl, CaCl₂, MgCl₂ are salts necessary for the survival of living organisms. KH₂PO₄ is a buffer which makes it possible to maintain a constant pH.

Media 1 and 2, once prepared, are distributed in 250 cm³ Erlenmeyer flasks. They are used in liquid form or gelled form (for Medium 2) incorporating agar at 3% by mass (gelling polysaccharide). The latter is poured into Petri dishes and is used to carry out the counts during the assay. The media are sterilized by autoclave (120° C. for 20 minutes) then kept in cold storage. The glucose is autoclaved separately in order to prevent its caramelization with the medium.

Assembly

The assembly is illustrated in FIG. 1. It is constituted by an air compressor/aerosol generator (Diffusion Technique Française—Saint-Etienne—France), connected to a nebulizer (Atomisor NL 11). A gas filter with a pore size of 0.22 μm (Gelman) is placed between the generator and the nebulizer in order to prevent parasitic bacterial contaminations.

The cell is a filter support in two parts, made of glass, comprising an air inlet and outlet. The membrane is inserted in a sterile fashion between two rubber gaskets covering the ground glass joints of the cell. The assembly is held together by clamps.

The cell outlet is connected to a liquid trap (50 cm³ of physiological serum) in order to dissolve by bubbling the viruses which have passed through the membrane. The flow of residual air passes through a second liquid trap containing Javel water in order to neutralize any traces of virus which were not dissolved.

The whole of the assembly is autoclaved, taken to pieces then assembled in a sterile fashion under a laminar flow hood previously sterilized under UV.

Procedure for the Filtration Experiments

The experiments follow the same procedure:

Manufacturing of a Viral Solution:

In a first phase, E. coli is inoculated with an inoculation loop into 150 cm³ of medium 1 at 37° C. The development of the growth of the bacterium is monitored by a UV/visible spectrophotometer. When the OD at 580 nm reaches 0.5 (i.e. a cell population of 2.1×10⁸ cells/ml), 18 μl of a reference solution of phages (containing 10¹² phages/ml) is diluted in 182 μl of physiological serum. 20 μl of this solution is then inoculated into the medium 1 containing E. coli. The quantity of phages injected must always be proportional to the cell population. After culture overnight, 130 ml of medium is centrifuged at 3000 rpm for 10 minutes in order to eliminate the bacteria. The supernatant is filtered on Millex type HA (Millipore) with a pore size of 0.45 μm. The filtrate is recovered in order to serve as stock solution to be nebulized.

Filtration Experiment:

5 cm³ of the phage stock solution is nebulized for approximately 1 hour (air flow rate: 8 L/h). The assembly described previously made it possible to carry out the following experiments:
With no membrane, in order to determine the virus losses due to the assembly.
With the 3 layers of surgical mask, in order to determine its viral retention.
With a non-functionalized fibre bed.
With a fibre bed functionalized by 4.4% PEI (g/g) at pH=8 in 0.2M NaCl.
With a fibre bed functionalized by 4.4% PEI (g/g) at pH=6 in 0.2M NaCl.
With a fibre bed. functionalized by 0.44% PEI (g/g) at pH=6 in 0.2M NaCl.

Titration of the Viruses

At the end of the experiment, 0.4 ml of the solution placed at the cell outlet are removed and diluted in 3.6 cm³ of physiological serum. This solution is diluted successively up to a dilution of 10⁻⁸. 0.25 ml of each dilution is then transferred to empty sterile tubes to which is added 0.4 ml of a suspension of E. coli in exponential growth phase previously cultured in 150 ml of medium 2 (reference OD₅₈₀=0.8). These tubes are then placed at 37° C. for 30 min in order to allow the phages to contaminate E. Coli. 3 ml of soft agar (medium 2 containing 0.6% agar) at 40° C. is then added to each of the tubes. The contents of each tube are spread over Petri dishes containing a solid agar (3% agar) of medium 2. The dishes are placed at 37° C. for 24 hours before counting the lysis plaques.

The stock solution is also titrated in the same way (with a dilution range up to 10⁻¹¹) and serves as a reference for each experiment.

The loss factor is equal to the number of viruses per cm³ of stock solution divided by the number of phages per -cm³ of receiving solution after filtration.

2—Results and Discussions

Filtration in the Cell with No Membrane

The filtration experiment with no membrane serves to evaluate the phage loss due to the nebulization, when the phage passes into the cell and during its recovery by bubbling through the physiological serum solution. 0.72×10¹⁰ viruses per cm³ were found for a nebulized stock solution containing 1.18×10¹⁰ viruses per cm³. The loss factor for the assembly is therefore 1.6.

Filtration through the Commercial Mask

The effectiveness of the viral retention of the surgical mask constituted by the 3 layers was tested. 0.56×10¹⁰ viruses per cm³ were found for a nebulized stock solution containing 2.88×10¹⁰ viruses per cm³. The loss factor. for the mask is therefore 5.2. Given the loss factor specific to the assembly alone, the commercial surgical mask is therefore totally ineffective vis-à-vis the viruses.

Filtration through the Non-Functionalized Fibre Bed

The effectiveness of the viral retention of the fibre bed without treatment was measured. 0.43×10¹⁰ viruses per cm³ diffused through this untreated fibre bed for a nebulized stock solution containing 1.22×10¹⁰ viruses per cm³. The loss factor due to the untreated fibre bed (i.e. the untreated layer 3) is therefore 2.9. This factor is less than that of the 3-layer surgical mask which is logical, as the two polypropylene layers intended for bacterial filtration also very slightly filter out the viruses.

Filtration through the Fibre Bed Functionalized by 4.4% PEI (g/g) in 0.2M NaCl at pH=8

Measurement of the viral retention of the fibre bed treated with PEI shows that 1.44×10³ viruses per cm³ have diffused for a nebulized stock solution containing 0.5×10¹⁰ viruses per cm³. The loss factor due to the untreated layer 3 is therefore 3.5×10⁶. The treatment with 4.4% PEI (g/g) in 0.2M NaCl medium at pH=8 is therefore very effective at slowing down the diffusion of the viruses. However, the treatment does not make it possible to make the layer a complete barrier vis-à-vis the viruses.

Filtration through the Fibre Bed Functionalized by 4.4% PEI (g/g) in 0.2M NaCl at pH=6

A nebulized stock solution containing 0.7×10¹⁰ viruses per cm³ is filtered through a fibre bed functionalized by 4.4% PEI (g/g) in 0.2M NaCl at pH=6. Measurement of the viral retention by this layer shows that no virus is detected after filtration. The retention of the viruses by the treated layer 3 is therefore total. This layer has become a complete barrier vis-à-vis the viruses due to our treatment. The PEI is therefore a very effective polymer for making the filtration system a barrier vis-à-vis the viruses.

It was verified, by TOC (total organic carbon) assay, that the PEI did not become detached from the support during respiration after 5 hours of use. Respiration was simulated by diffusion of air at 8 L/min.

Filtration through the Fibre Bed Functionalized by PEI at 0.44% (g/g) in 0.2M NaCl at pH=6

The concentration of PEI binded to the fibre bed was diluted 10 times in order to verify its effectiveness in a more dilute solution. Measurement of the viral retention shows that 5.5×10⁵ viruses per cm³ passed through the filter for a nebulized stock solution containing 5.5×10⁷ viruses per cm³. The loss factor is therefore 100. Treatment with 0.44% PEI (g/g) is therefore clearly less effective than treatment with 4.4% (g/g).

1. Fibre bed, preferably negatively charged, coated with at least one cationic polymer, the nitrogen atoms of which are not substituted by alkyl groups and the nitrogen atoms of which are not quaternized, and having a protonation level of at least 20%.

2. Fibre bed according to claim 1, coated on one or other of its surfaces, with a film of cationic polymer having a thickness of 0.5.10⁻³ μm to 5 μm, preferably 0.001 to 1 μm, very preferably 0.001 to 0.01 μm.

3. Fibre bed according to claim 1, wherein the positive charge density is 1×10⁻⁶ to 1×10⁻⁸ moles of charge per cm² of fibre bed or 1×10⁻⁵ to 1×10⁻³ meq per cm² of fibre bed, preferably 1×10⁻⁷ mol of charge per cm² of bed or 1×10⁻⁴ meq per cm² of fibre bed.

4. Fibre bed according to claim 1, characterized in that said polymer comprises cationic groups on its main chain and/or on its side chain or chains.

5. Fibre bed having antiviral properties according to claim 1, characterized in that said polymer is polyethylene-imine.

6. Antiviral filter comprising at least one fibre bed as described in claim 1.

7. Antiviral filter according to claim 6,
furthermore comprising a fibre bed comprising a pore size equal to or less than 0.2 μm.

8. Antiviral filter according to claim 6, characterized in that said fibre bed has a pore size equal to or less than 0.2 μm.

9. Air purifier, air conditioner or air humidifier comprising at least one filter as described in claim 6.

10. Medical device comprising at least one filter as described in claim 1, or at least one fibre bed as described in any one of claims 1 to 5.

11. Medical device according to claim 10,
which is a surgical mask, a dressing or a protective garment such as overalls.

12. Method for the manufacturing of a fibre bed with antiviral properties comprising contacting of said fibre bed with a solution of cationic polymer as described in claim 1, then a drying stage.

13. Method for the manufacturing of a fibre bed with antiviral properties according to claim 12, characterized in that the concentration of the polymer in the solution is comprised between 0.1 and 200 g.l⁻¹, preferably between 1 and 100 g.l⁻¹, more preferably between 20 and 80 g.l⁻¹ and very preferably between 40 and 50 g.l⁻¹.

14. Method for the manufacturing of a fibre bed with antiviral properties according to claim 1 characterized in that the pH of the polymer solution is preferably acid, preferably comprised between 3 and 6.5, and very preferably the pH of the polymer solution is 6.

15. Medical device comprising at least one fibre bed as described in claim 1.

16. Fibre bed according to claim 2, wherein the positive charge density is 1×10⁻⁶ to 1×10⁻⁸ moles of charge per cm² of fibre bed or 1×10⁻⁵ to 1×10⁻³ meq per cm² of fibre bed, preferably 1×10⁻⁷ mol of charge per cm² of bed or 1×10⁻⁴ meq per cm² of fibre bed.

17. Fibre bed according to claim 2, characterized in that said polymer comprises cationic groups on its main chain and/or on its side chain or chains.

18. Fibre bed according to claim 3, characterized in that said polymer comprises cationic groups on its main chain and/or on its side chain or chains.

19. Fibre bed having antiviral properties according to claim 2, characterized in that said polymer is polyethylene-imine.

20. Fibre bed having antiviral properties according to claim 3, characterized in that said polymer is polyethylene-imine.
